(19) United States
(12) Patent Application Publication
(10) Pub. No.: US 2025/0082463 A1
(43) Pub. Date: Mar. 13, 2025

(54) PRELOADED INTRAOCULAR LENS INJECTOR AND METHOD OF USE THEREOF

(71) Applicant: ICARES Medicus, Inc., Hsinchu (TW)

(72) Inventors: Ming-Yen SHEN, Hsinchu (TW); Chun-Ming LIN, Hsinchu (TW); William LEE, Chelsea, MA (US)

(73) Assignee: ICARES Medicus, Inc., Hsinchu (TW)

(21) Appl. No.: 18/728,848

(22) PCT Filed: Jan. 18, 2023
(86) PCT No.: PCT/US2023/060839

§ 371 Date: Jul. 12, 2024

Related U.S. Application Data
(60) Provisional application No. 63/300,449, filed on Jan. 18, 2022.

(51) Int. Cl. A61F 2/16

(52) U.S. Cl. CPC A61F 2/167; A61F 2/1675

(57) ABSTRACT

The present disclosure provides a device and method for inserting an IOL into an eye of a patient. The IOL injector can be configured to preload an IOL into the lens cartridge of the injector without manual manipulation of the IOL by the nurse and/or physician during the procedure. The injector can be configured to properly orient and align the IOL within the injector and maintain proper alignment throughout delivery of the IOL to the eye of a patient and thereby ensuring that the IOL is properly positioned and oriented at a predetermined location in the eye.

The application claims benefit of priority under 35 U.S.C. § 119(e) of U.S. Ser. No. 63/300,449, filed Jan. 18, 2022, the content of which is incorporated by reference in its entirety.

### FIELD

The present disclosure relates generally to ophthalmic surgical devices and methods, and more particularly to a device and method for inserting an intraocular lens (IOL) into an eye, wherein the IOL may be conveniently preloaded in and packaged together with the injector device.

### BACKGROUND

Intraocular lens (IOL) injectors are devices designed to insert an IOL into the eye of a patient. Such lenses are surgical implants designed to augment or replace the natural lens of the eye and are used to treat certain diseases of the eye. For example, the impairment of vision caused by cataracts is often treated by the surgical removal and replacement of the eye's lens. During such a surgery a small 1-4 mm incision is formed in the patient's eye. The surgeon then uses a tool, inserted into the eye through the incision, to emulsify (break up) and remove the eye's natural lens. Once removed an artificial intraocular lens is inserted in the eye.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL injectors must therefore be designed in such a way as to permit the easy passage of the IOL through the device and into the eye, yet at the same time does not damage the delicate IOL in any way. Should the IOL be damaged during delivery into the eye, the surgeon will most likely need to extract the damaged IOL from the eye and replace it with a new IOL, a highly undesirable surgical outcome.

It is also important that the IOL be expelled from the tip of the IOL injector and into the eye in a predictable orientation and manner. Should the IOL be expelled from the tip too quickly or in the wrong orientation, the surgeon must further manipulate the IOL in the eye, which could result in trauma to the surrounding tissues of the eye. It is therefore highly desirable to have an IOL injector device that allows for precise loading of the IOL into the IOL injector and that will pass and expel the IOL from the injector tip and into the eye in a controlled, predictable, and repeatable manner.

To ensure controlled expression of the IOL through the tip of the IOL injector, the IOL must first be loaded into the IOL injector. The loading of the IOL into the injector is therefore a precise and very important step in the process. Incorrect loading of an IOL into the injector is oftentimes cited as the reason for a failed IOL delivery sequence. Many IOL injector devices on the market today require the IOL to be loaded into the injector at the time of surgery by the attending nurse and/or surgeon. Due to the delicate nature of the IOL, there is a risk that the nurse and/or surgeon will inadvertently damage the IOL and/or incorrectly load the IOL into the injector device resulting in a failed implantation. Direct handling and/or loading of the IOL into the injector by the nurse and/or surgeon is therefore undesirable.

Accordingly, there remains a need for an IOL injector device and method which remove the need for direct handling of the IOL by the nurse and/or surgeon and which generally simplifies operation of the IOL injector device and IOL delivery process.

### SUMMARY

The present disclosure addresses the deficiencies of conventional injectors by providing an intraocular lens (IOL) injector preloaded with an IOL in the unfolded state, wherein the injector is configured to automatically fold and properly align the IOL during delivery of the IOL without relying on the skill of the nurse and/or surgeon.

In some embodiments, an intraocular lens (IOL) injector is provided comprising: an injector body elongated along a longitudinal direction, the injector body having an injector body lumen disposed along the longitudinal direction; a lens cartridge in operable connection with the injector body, the lens cartridge having a lens cartridge lumen configured to receive an IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL; an injector tip in operable connection with the lens cartridge, the injector tip having an injector tip lumen disposed along the longitudinal direction and terminating in a distal opening, wherein the injector tip lumen is coaxial with the injector body lumen; and a plunger having an elongated shaft, the elongated shaft being slidably disposed within the injector body lumen from an undeployed position to a deployed position, wherein the plunger is configured to contact the IOL in the lens cartridge lumen, push the IOL along the longitudinal direction through the injector tip lumen to fold the IOL and inject the IOL out of the distal opening when the plunger is transitioned to the deployed position.

In some embodiments, a method of implanting an intraocular lens (IOL) is provided, the method comprising: providing an IOL injector preloaded with an IOL, the injector comprising: an injector body elongated along a longitudinal direction, the injector body having an injector body lumen disposed along the longitudinal direction; a lens cartridge preloaded with the IOL and in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL; an injector tip in operable connection with the lens cartridge, the injector tip having an injector tip lumen disposed along the longitudinal direction and terminating in a distal opening, wherein the injector tip lumen is coaxial with the injector body lumen; and a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position; and transitioning the plunger from the undeployed position to the deployed position, thereby ejecting the IOL from the distal opening of the injector tip lumen into an eye of a patient, the IOL being in a folded state while exiting the distal opening.

In some embodiments, a method of implanting an intraocular lens (IOL) is provided, the method comprising: providing an IOL injector, the injector comprising: an injector body elongated along a longitudinal direction, the injector body having an injector body lumen disposed along the longitudinal direction; a lens cartridge in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL; an injector tip in operable connection with the lens cartridge, the injector tip having an injector tip lumen disposed along the longitudinal direction and terminating in a distal opening, wherein the injector tip lumen is coaxial with the injector body lumen; and a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position; inserting the IOL into the lens cartridge lumen; and transitioning the plunger from the undeployed position to the deployed position, thereby ejecting the IOL from the distal opening of the injector tip lumen into an eye of a patient, the IOL being in a folded state while exiting the distal opening.

In some embodiments, a kit comprising an intraocular lens (IOL) injector and an IOL is provided, the IOL comprising: an injector body elongated along a longitudinal direction, the injector body having an injector body lumen disposed along the longitudinal direction; a lens cartridge in operable connection with the injector body, the lens cartridge having a lens cartridge lumen configured to receive an IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL; an injector tip in operable connection with the lens cartridge, the injector tip having an injector tip lumen disposed along the longitudinal direction and terminating in a distal opening, wherein the injector tip lumen is coaxial with the injector body lumen; and a plunger having an elongated shaft, the elongated shaft being slidably disposed within the injector body lumen from an undeployed position to a deployed position, wherein the plunger is configured to contact the IOL in the lens cartridge lumen, push the IOL along the longitudinal direction through the injector tip lumen to fold the IOL and inject the IOL out of the distal opening when the plunger is transitioned to the deployed position.

### BRIEF DESCRIPTION OF THE DRAWINGS

The present application can be best understood by reference to the following description taken in conjunction with the accompanying figures included in the specification.

FIGS. 1A-1B is a perspective view of an IOL injector, with an expanded view of the distal end of the IOL injector, according to some embodiments of the present disclosure;

FIGS. 2A-2B is a front view of the IOL injector, with an expanded view of the distal end of the IOL injector, according to some embodiments of the present disclosure;

FIG. 3 is a back view of the IOL injector, according to some embodiments of the present disclosure;

FIGS. 4A-4B is a side view of the IOL injector, with an expanded view of the distal end of the IOL injector, according to some embodiments of the present disclosure;

FIGS. 5A-5B is a side view of the other side of the IOL injector, with an expanded view of the distal end of the IOL injector, according to some embodiments of the present disclosure;

FIGS. 6A-6B is a top view of the IOL injector, with an expanded view of the distal end of the IOL injector, according to some embodiments of the present disclosure;

FIGS. 7A-7B are bottom views of the IOL injector, with an expanded view of the distal end of the IOL injector, according to some embodiments of the present disclosure;

FIGS. 8A-8B is a side view and a top view of the elongated shaft of the plunger, respectively, with an expanded view of the distal tip of the plunger, according to some embodiments of the present disclosure;

FIGS. 9A-9K are various views of the lens cartridge of the IOL injector, according to some embodiments of the present disclosure; and

FIGS. 10A-10B illustrate lens delivery forces of the IOL injector, according to some embodiments of the present disclosure.

### DETAILED DESCRIPTION

In various aspects, the present disclosure provides a device and method for inserting an IOL into an eye of a patient. The injector is configured to properly orient and align the IOL within the injector and maintain proper alignment throughout delivery of the IOL to the eye of a patient and thereby ensuring that the IOL is properly positioned and oriented at a predetermined location in the eye.

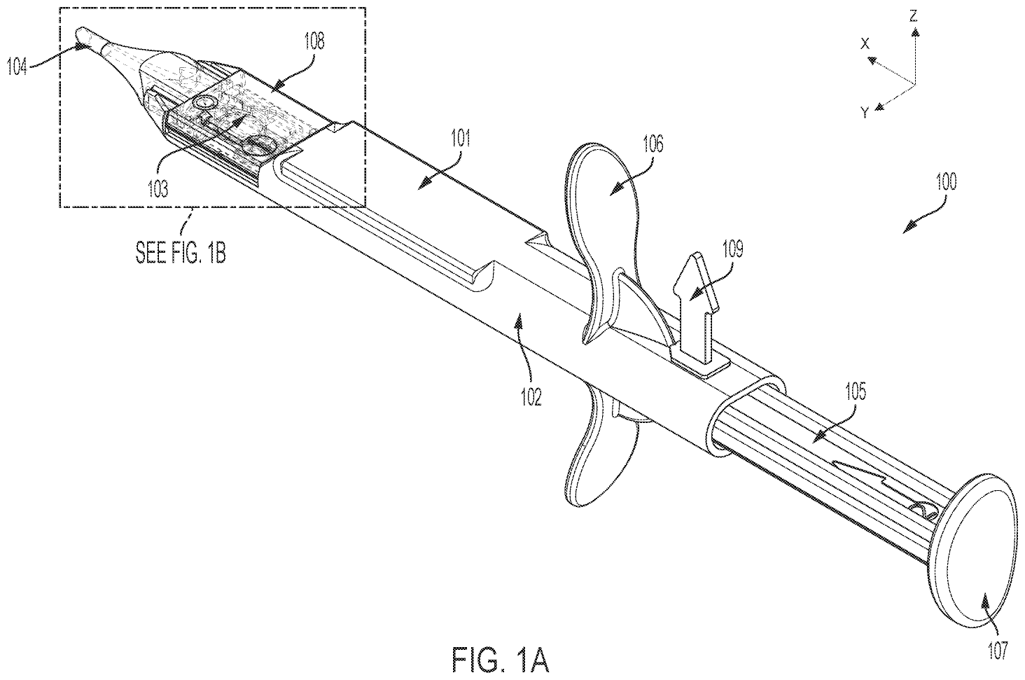

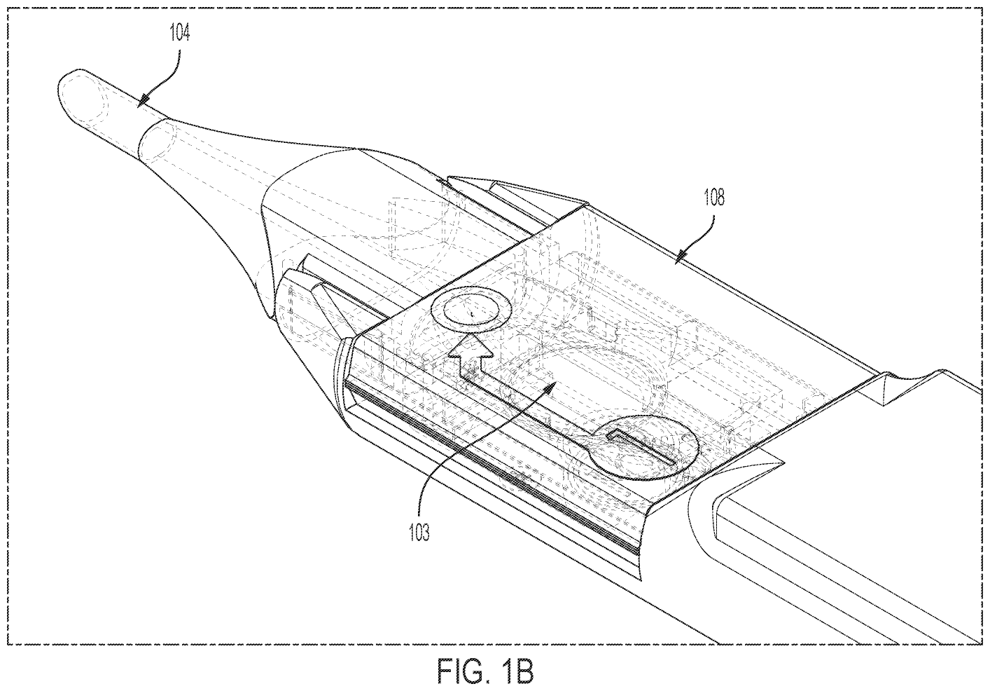

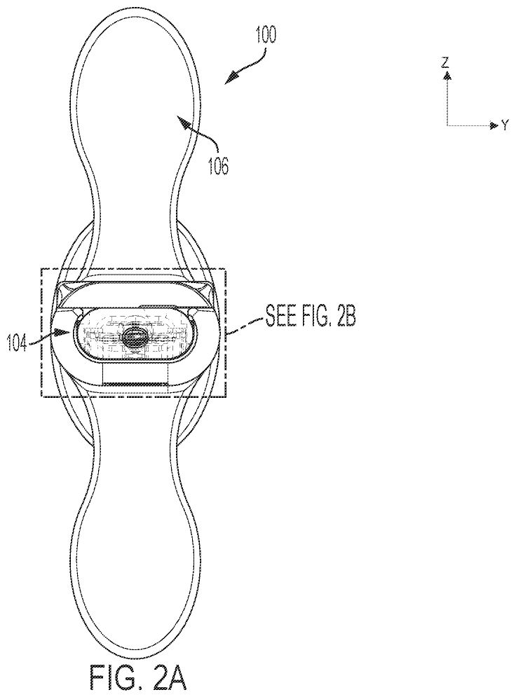

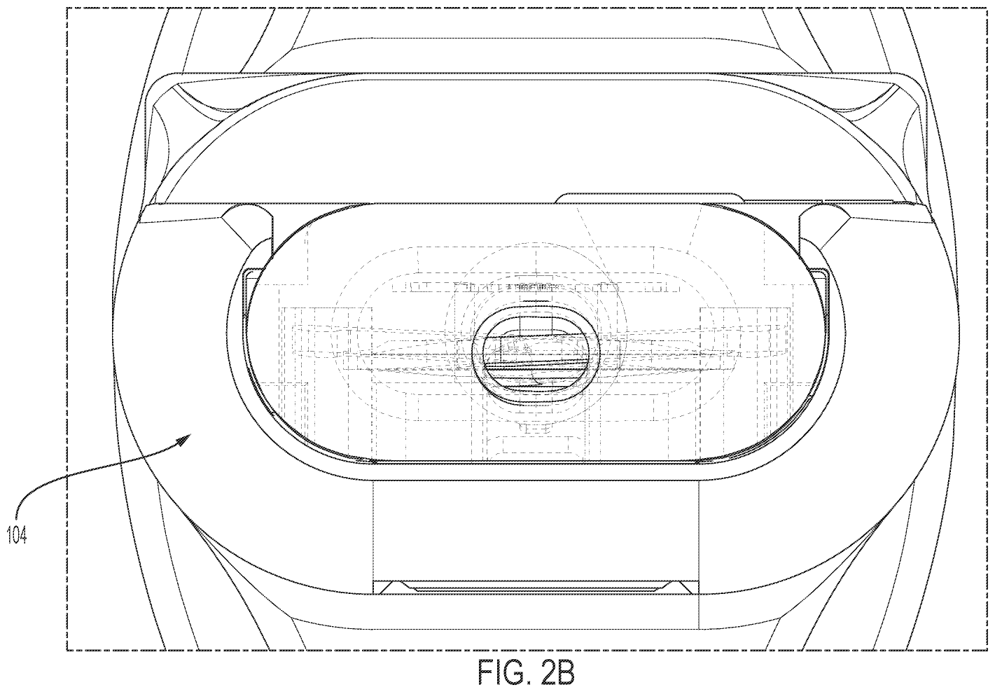

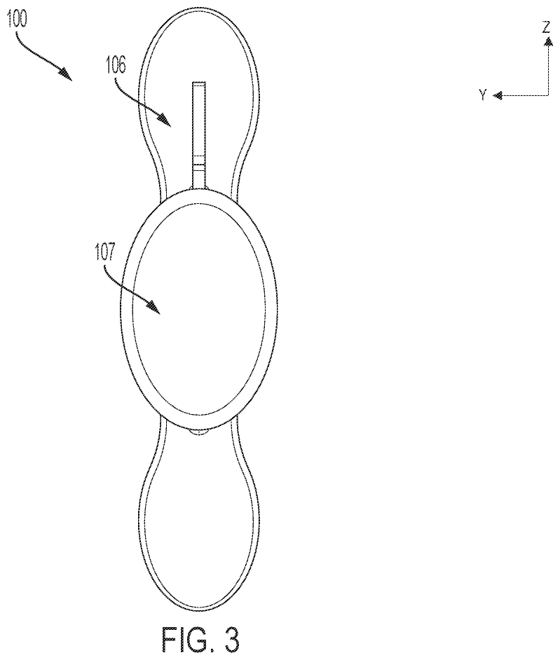

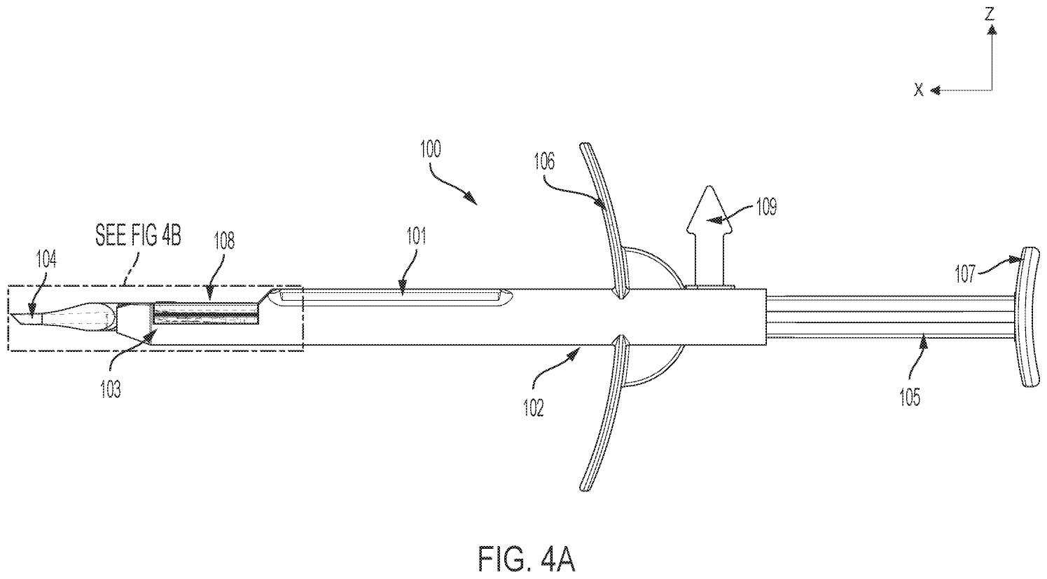

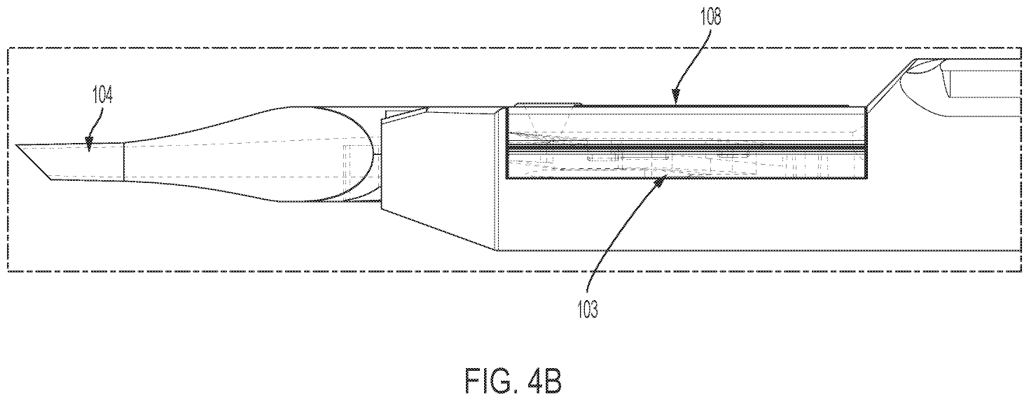

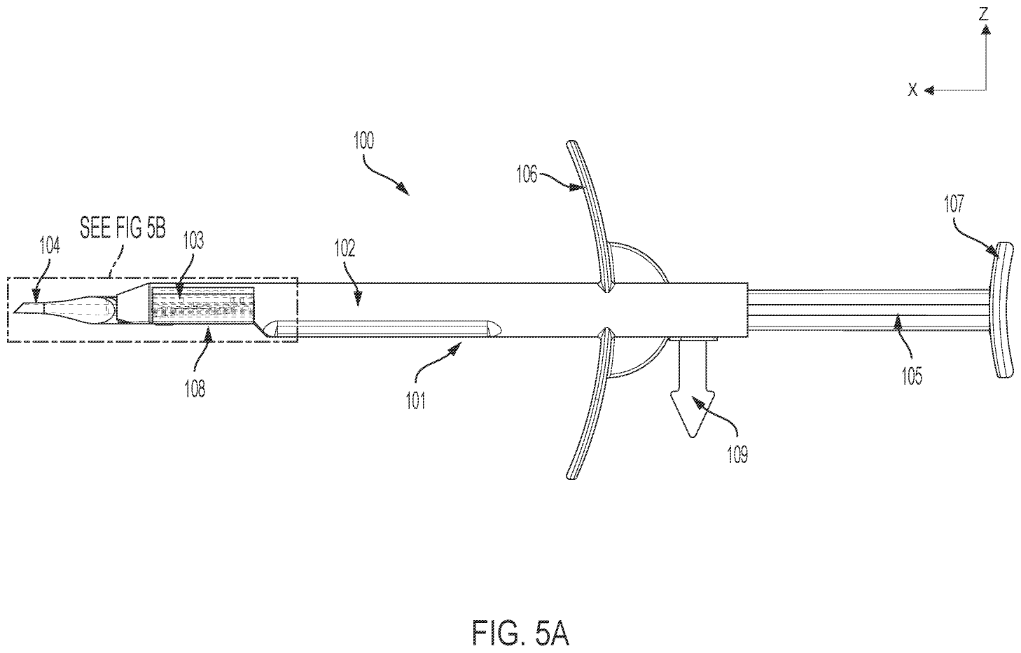

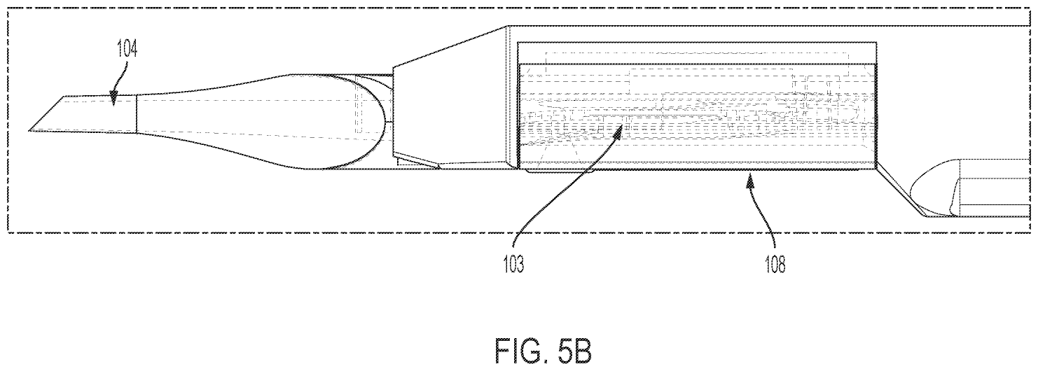

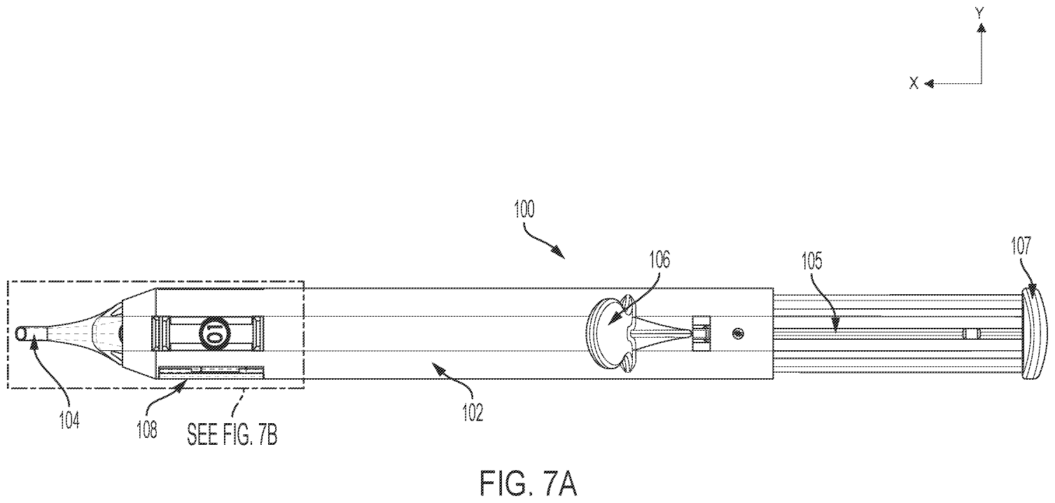

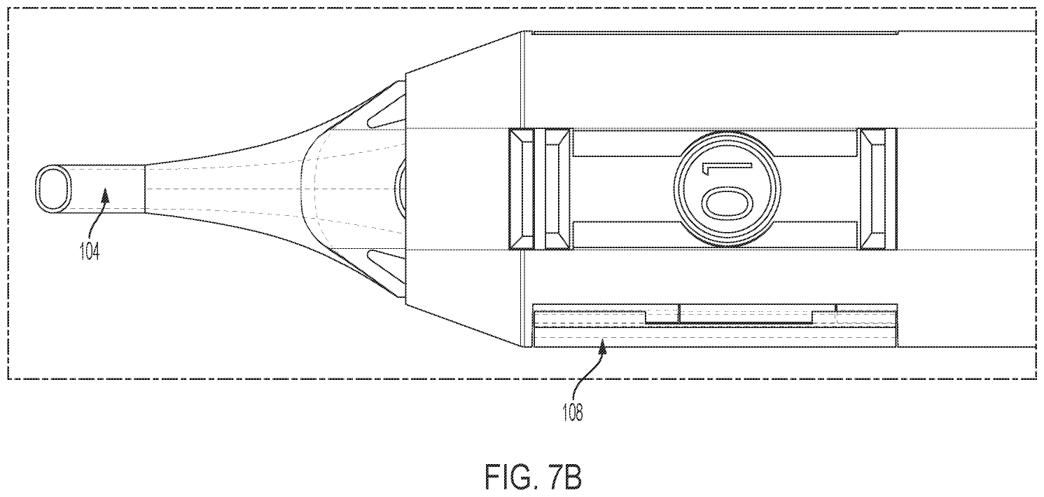

Referring to FIGS. 1A-7B, an IOL injector 100 according to some embodiments of the present disclosure can include a label platform 101, an injector body 102, a lens cartridge 103 in operable connection with the injector body 102, an injector tip 104 in operable connection with the lens cartridge 103, a plunger 105, a finger hold flange 106, a proximal thumb press 107, and a viewing window 108. In some embodiments, a stopper 109 (shown as an arrow in the illustrated embodiment) can also be included in the disclosed IOL injector 100. In some embodiments, X in the coordinate system can indicate a longitudinal axis, Y can indicate a lateral axis, and Z can indicate a vertical axis.

The label platform 101 can be a flat surface on the injector body 102, which allows a label to be securely attached, such that a user can read the printed information on the label with ease while operating the device. The viewing window 103 can be a flat transparent surface on top of the lens cartridge 103 and the injector tip 104, which provides the user with an unobstructed view of the entire cartridge and tip lumen along the longitudinal axis, thus allowing the user to accurately identify and locate the positioning of the IOL, its haptics, and its current folding status prior, during, and after lens pushing. The finger hold flange 106 and proximal thumb press 107 can be configured with contoured grip surfaces, which allow for a good ergonomic fit with the user's hands. In some embodiments, the disclosed IOL injector 100 can include a stopper 109. The stopper 109 can be used to prevent the plunger from moving in the package and during transportation, such that the preloaded IOL can be kept in an unfolded state before use.

The plunger 105, the injector body 102, the lens cartridge 103 and the injector tip 104 can be arranged along a longitudinal axis of the IOL injector 100 with the plunger 105 being disposed on the proximal end of the injector and the injector tip 104 being disposed on the distal end of the injector. In some embodiments, a lumen (not depicted) can traverse the entire length of the injector 100 such that an IOL can be loaded into the lens cartridge 103 and pushed by a distal tip of the elongated shaft of the plunger 105 along the longitudinal axis through the injector tip 104 into a patient's eye, when the plunger 105 is advanced distally from an undeployed position to a deployed position.

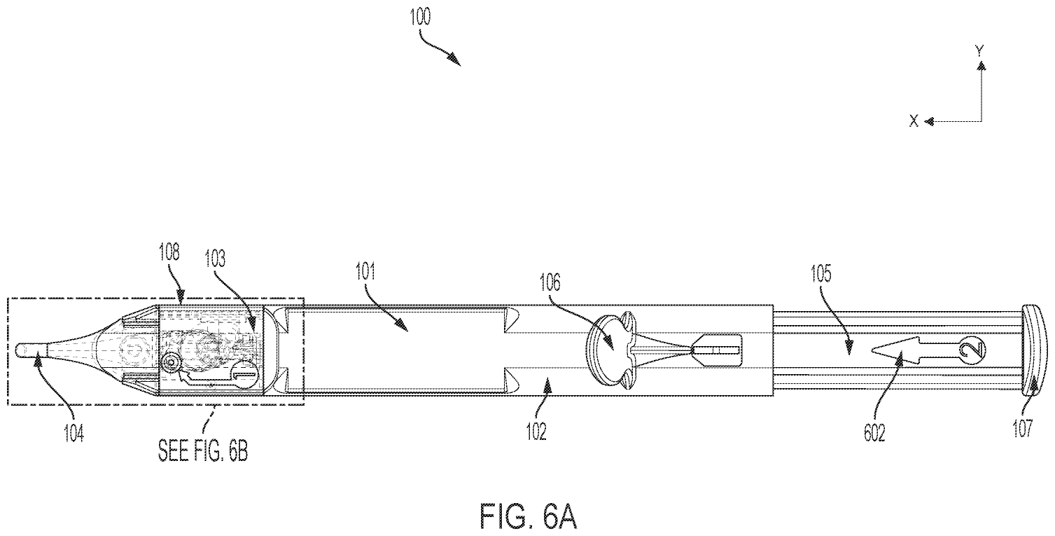

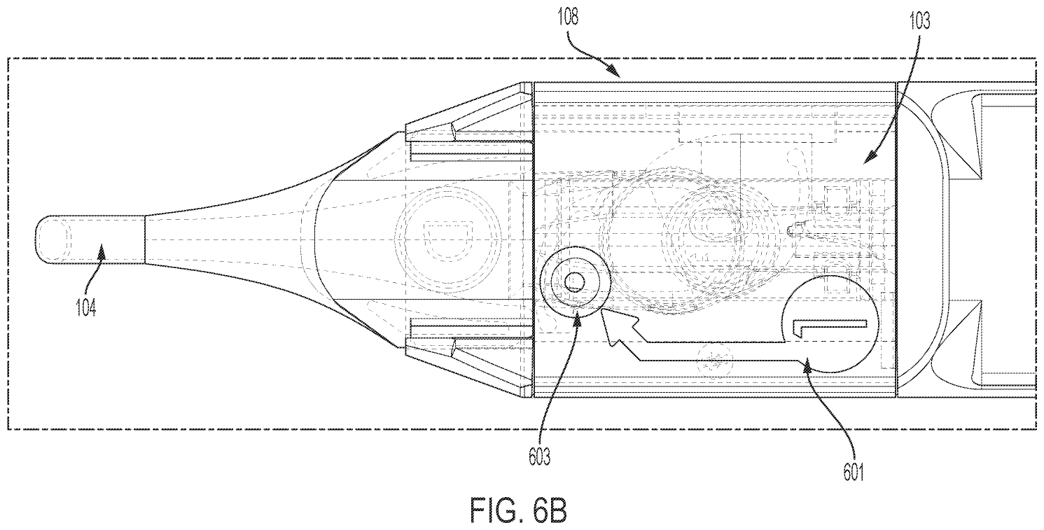

FIGS. 6A-6B is a top view of the disclosed IOL injector 100, with an expanded view of the distal end of the IOL injector. In some embodiments, the disclosed IOL injector 100 can include a first instruction indicator 601 and a second instruction indicator 602, shown as arrows in the illustrated example, which can provide a facile and clear set of instructions to the doctor as to how the inventive IOL injector should be operated. During the insertion of the IOL, a fluid such as water, a viscoelastic substance, a balanced salt solution (BSS), and the like can be used to facilitate the passing of the IOL through the IOL injector 100. In some embodiments, the fluid can be preloaded into the lens cartridge 103. In some embodiments, the fluid can be added into the lens cartridge 103 through an aperture 603. The first instruction indicator 601 indicates the position (the aperture 603) on the lens cartridge 103 where the fluid should be added. The second instruction indicator 602 indicates the correct direction the plunger 105 should be transitioned for purposes of injecting the preloaded IOL into the patient's eye. In some embodiments, the disclosed IOL injector 100 can be packaged in a way that the plunger 105 is placed in an undeployed position such that the distal end of the plunger 105 has not entered the lens cartridge 103. Accordingly, once a user opens the package, the label platform 101, the first instruction indicator 601 and the second instruction indicator 602 can provide clear information and operation steps for the use of the IOL injector 100, such that the user can read the printed information and follow the suggested steps of operations to successfully inject the IOL into the patient's eye.

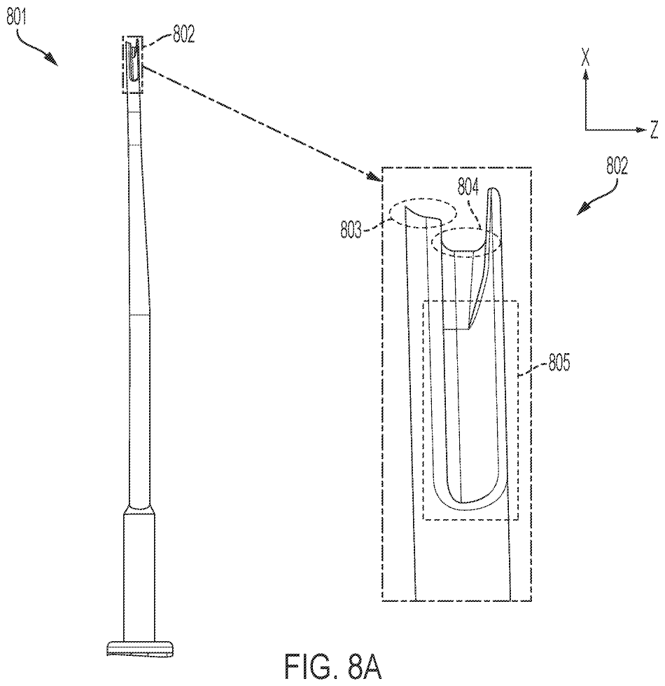

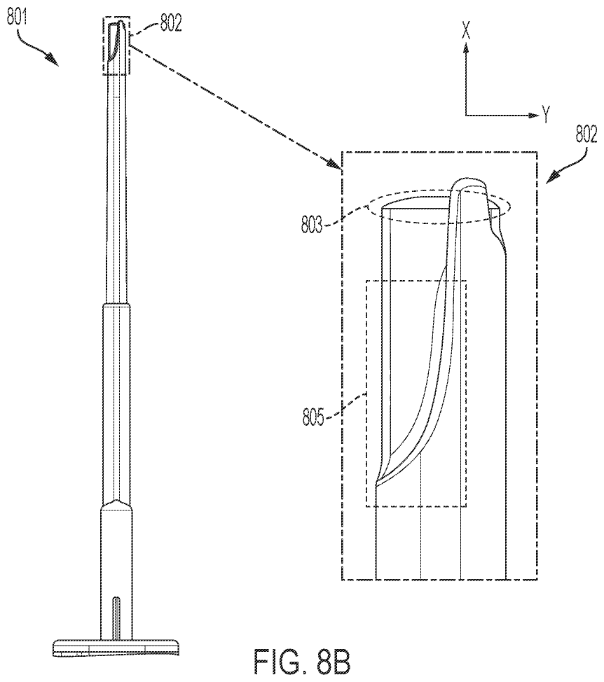

FIG. 8A is a side view of an elongated shaft 801 of the plunger 105, with an expanded view of a distal tip 802 of the plunger. FIG. 8B is a top view of the elongated shaft 801 of the plunger 105, with an expanded view of the distal tip 802 of the plunger. As part of the plunger stability mechanism, as it is advanced distally from an undeployed position to a deployed position, an angled distal plunger design with a vertical shift (−Z direction) from the center of the plunger is provided, which enables the plunger to maintain constant attachment to the floor of the lens cartridge 103 and the injector tip 104, such that the pushing action of the IOL is always conducted in a predictable manner.

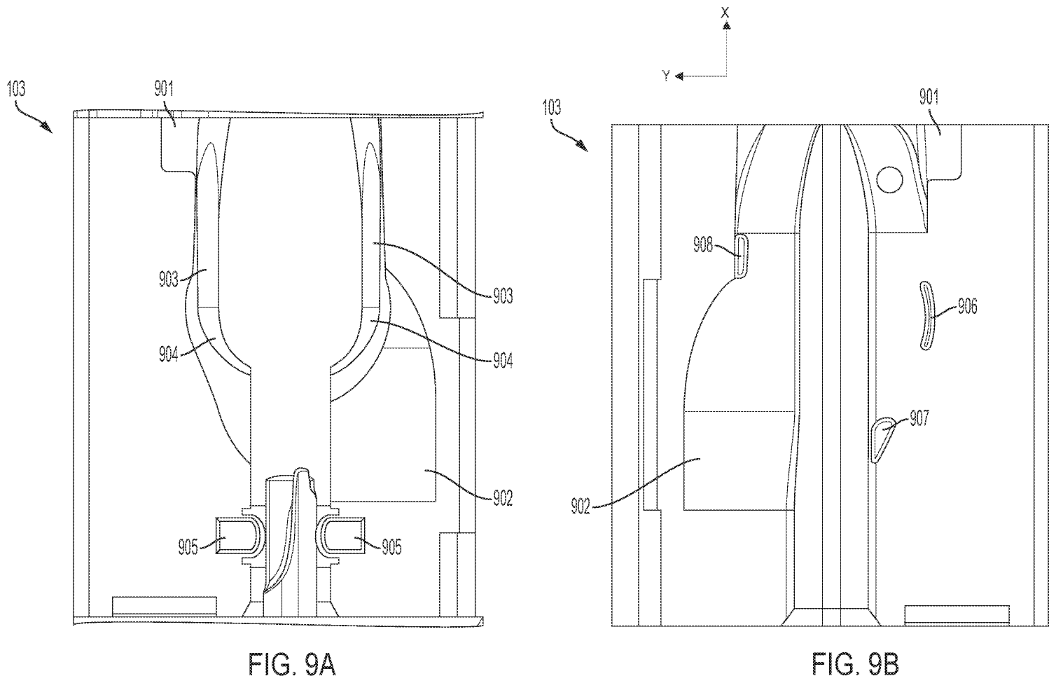

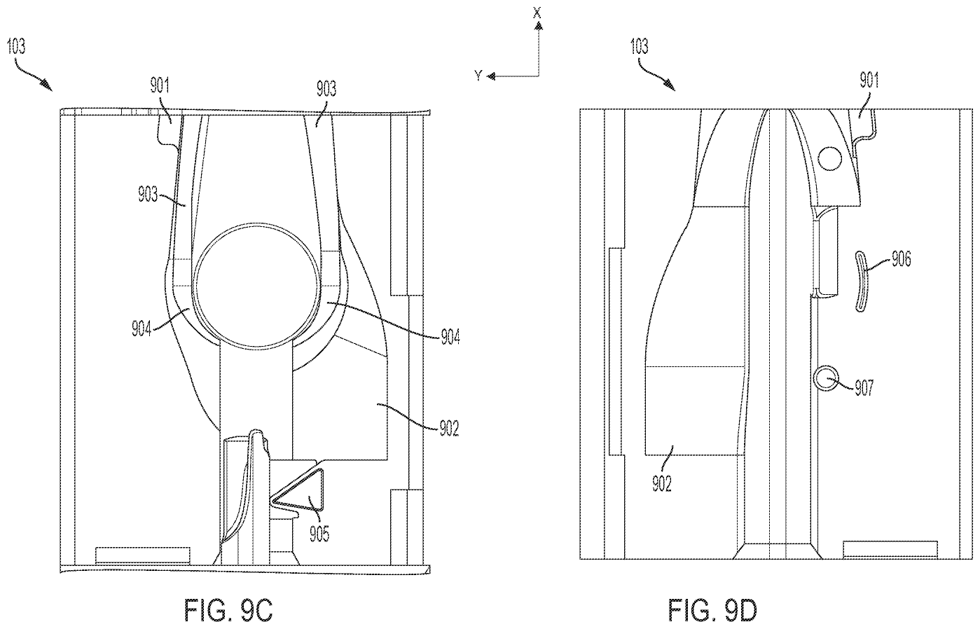

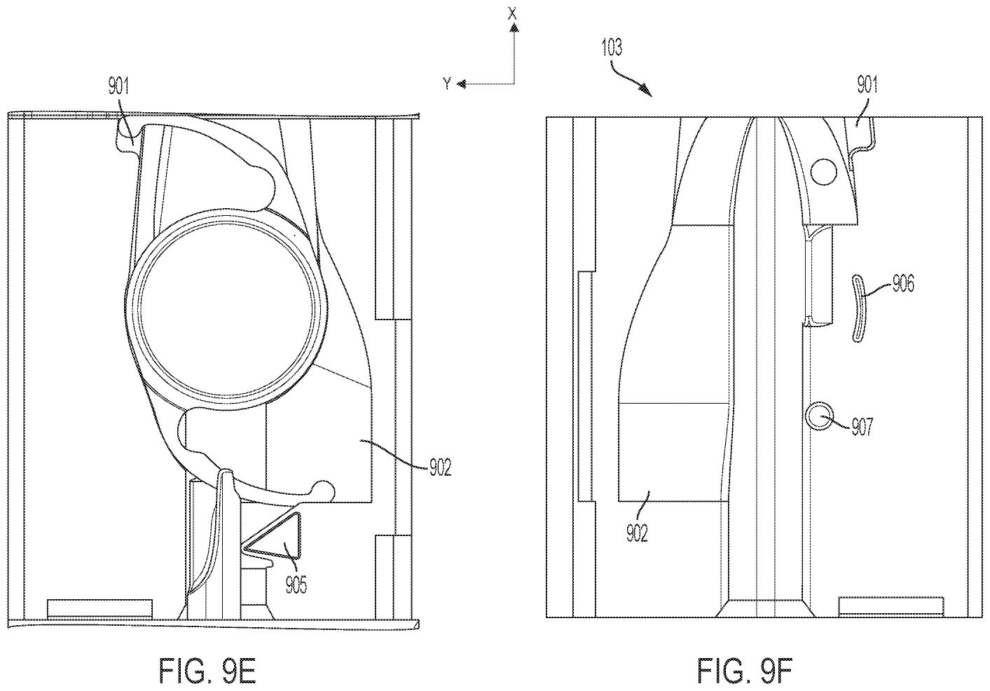

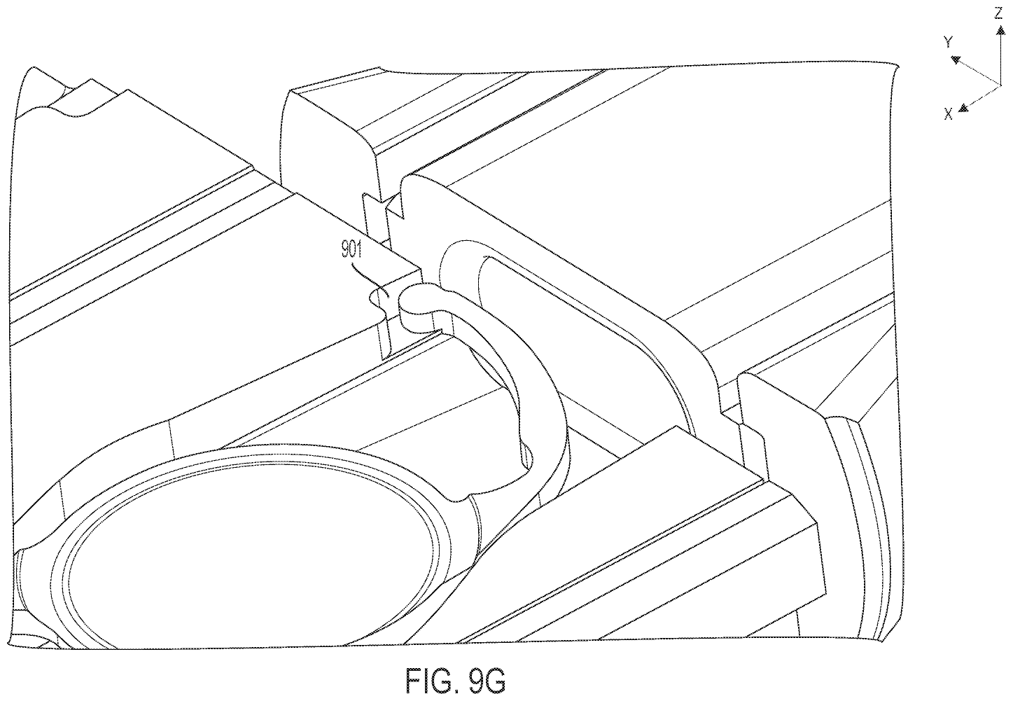

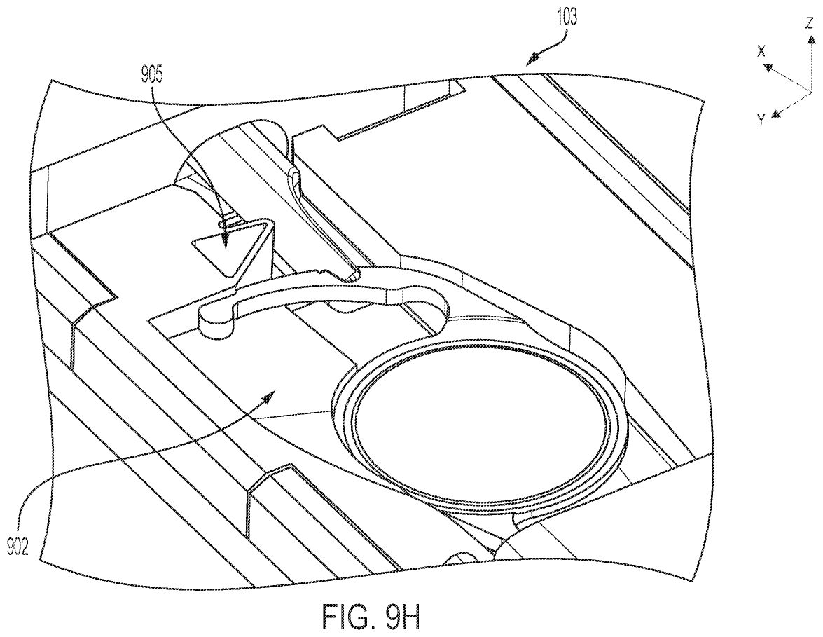

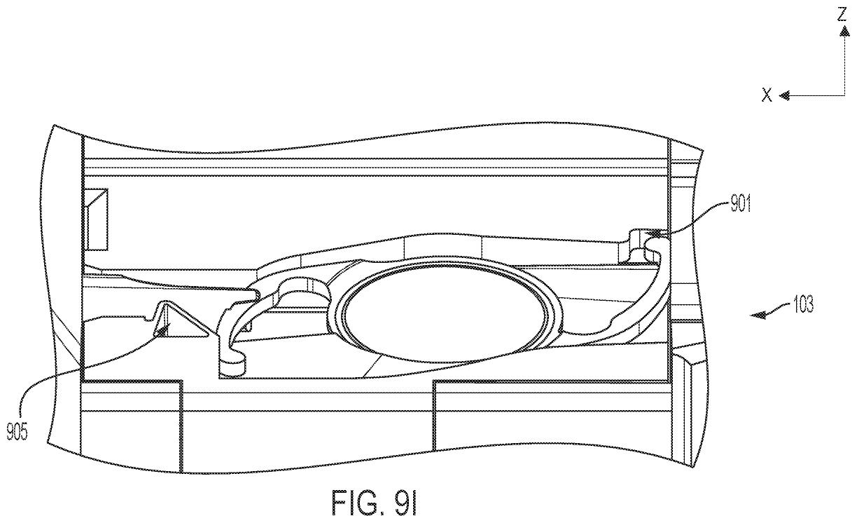

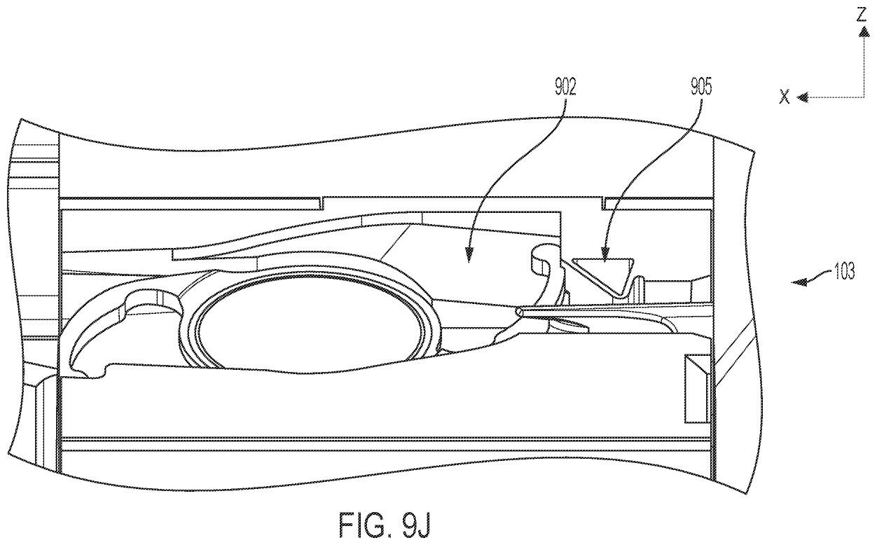

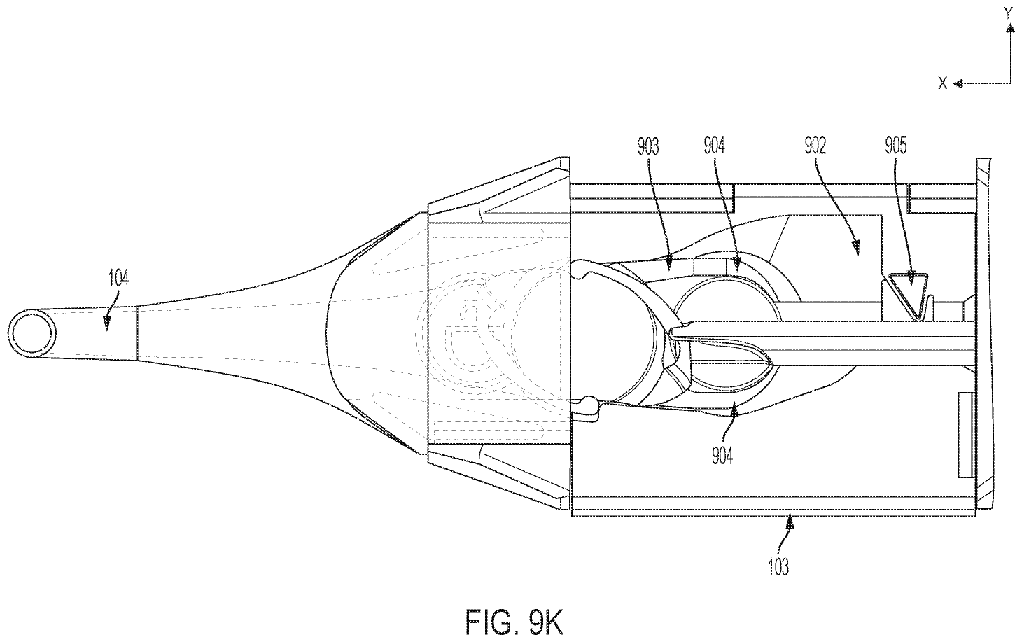

In some embodiments, where an IOL includes haptics, the injector can be configured to position one or more of the haptics on the optic region of the IOL in the correct orientation for delivery. In one embodiment, a properly aligned IOL has a leading haptic pushed toward the optic and a trailing haptic contacting the distal tip 802 of the plunger 105, as shown in FIG. 9E. As such, a properly oriented IOL has the haptics “tagged” to the optical area. In some embodiments, it can be advantageous to construct the distal tip 802 using a material which will not damage the IOL upon contact. In some embodiments, the distal tip 802 includes a polymeric material such as silicone, or other resiliently deformable polymer.

As shown by FIGS. 8A and 8B, an expanded view of the distal tip 802 reveals three distinctive features, including a curved surface 803, a middle cutout 804, and a lateral cutout 805. A distal plunger design composed of one or more of these three distinctive features can facilitate the IOL haptic folding and lens delivery, which can serve as a plunger hard-stick distal push mechanism. The curved surface 803 is located on the bottom (−Z direction) of the plunger that will push against the IOL's trailing optic edge. The curved surface 803 can essentially serve as the haptic pusher of the IOL to be delivered. In some embodiments, the curved surface may have a radius extending from a longitudinal axis of the plunger 105 to an edge of the curved surface 803 of about 0.5 mm, 0.55 mm, 0.60 mm, 0.65 mm, 0.70 mm, 0.75 mm, 0.80 mm, 0.85 mm, 0.90 mm, 0.95 mm, 1.00 mm. The middle cutout 804 is located in the middle of the plunger and can act as a holding mechanism for the trailing haptic, allowing the haptic to be grasped and pushed forward in the longitudinal direction X. The middle cutout 804 can essentially serve as the optic pusher of the IOL to be delivered. In some embodiments, the middle cutout 804 may have a width extending from a first side of the cutout to a second side of the cutout of about 0.20 mm, 0.25 mm, 0.30 mm, 0.35 mm, 0.40 mm, 0.45 mm, 0.50 mm, 0.55 mm, 0.60 mm, or more. The middle cutout 804 can work in conjunction with cartridge slope 902 of the lens cartridge (illustrated in FIGS. 9A-9F) to facilitate proper trailing haptic tucking onto the anterior optic surface. The lateral cutout 805 is a cutout feature on the left (−Y direction) side of the plunger, which provides clearance for the folded rear haptic to reside after completing haptic tucking. The lateral cutout 805 can essentially serve as the haptic tucking recess for the IOL to be delivered. In some embodiments, the lateral cutout may have a width extending from a first side of the cutout to a second side of the cutout of about 0.50 mm, 0.55 mm, 0.60 mm, 0.65 mm, 0.70 mm, 0.75 mm, 0.80 mm, 0.85 mm, 0.90 mm, 0.95 mm, 1.00 mm, or more. The lateral cutout 805 is intended to reduce the internal stress exerted by the folded rear haptic onto the inner injector tip wall, thereby minimizing potential cracking of the injector tip.

FIGS. 9A-9K illustrate various views of a lens cartridge, according to some embodiments of the present disclosure. FIGS. 9A, 9C, and 9E are top views of the lens cartridge 103, according to some embodiments of the present disclosure. FIGS. 9B, 9D, and 9E are bottom views of the lens cartridge 103, according to some embodiments of the present disclosure. Several notable features can be included in the lens cartridge 103 design to facilitate IOL tucking while maintaining a proper and predictable position of the IOL during the storage and injection process. The features can include a cartridge cutout 901, a cartridge slope 902, a straight wall 903, a curved wall 904, a plunger bumper 905, and a group of roof nubs 906, 907, and 908.

The cartridge cutout 901 is a recessed cutout design located on the left (−Y direction) surface of the IOL chamber wall (e.g., a lateral direction transverse to the longitudinal axis of the injector) that houses the IOL's leading haptic distal end after the IOL is loaded into the IOL chamber. The height of the cartridge cutout 901 is raised slightly higher than the intended IOL's anterior optic surface after IOL loading (as illustrated at least in FIGS. 9G and 9I). For example, the height of cartridge cutout 901 may be about 0.20 mm, 0.25 mm, 0.30 mm, 0.35 mm, 0.40 mm, 0.45 mm, 0.50 mm, 0.55 mm, 0.60 mm, 0.65 mm, 0.70 mm, or more. The cartridge cutout 901 allows the leading haptic to be folded backwards by temporarily holding on to the distal end of the haptic from advancing forward and bending the haptic at the shoulder (optic and haptic connection area) while the IOL optic is traveling forward along the longitudinal axis left (−X direction). Once the IOL optic advances a predetermined distance (e.g., about 3 mm or more in the illustrated example), the holding of the distal end of the haptic will begin to release the haptic, allowing the leading haptic to swivel and bend onto the anterior surface of the IOL optic, thereby completing the leading haptic tucking (illustrated at least in FIG. 9K).

The cartridge slope 902 is a recessed cutout design located on the right (+Y direction) surface of the IOL chamber wall (e.g., the lateral direction) that houses the IOL's trailing haptic distal end after IOL is loaded into the IOL chamber. As shown at least in FIGS. 9H and 9J, the cartridge slope 902 has an upward (+Z direction) sloping ramp that angles from the proximal end of the cartridge towards the distal end of the cartridge, where the height begins from the IOL optic center towards IOL anterior optic surface. For example, the angle of the cartridge slope 902 may be about 0.5°, 1°, 2°, 3°, 4°, 5°, 6°, 7°, 8°, 9°, 10°, or more, extending in a direction from the proximal end towards to the distal end of the cartridge. The cartridge slope 902 has sufficient lateral and vertical clearance for the trailing haptic to swivel and fold onto the anterior surface of the optic. In addition, the ramp limits the height of the haptic prior, during, and after haptic pushing by the plunger's middle cutout 804, thereby ensuring the haptic travels in a predictable path towards the anterior surface of the optic. The cartridge slope 902, in conjunction with the plunger function of directly pushing on the trailing haptic, allows the trailing haptic to travel upward on the cartridge slope 902 to reach the anterior optic surface, thereby completing the haptic tucking in a predictable manner.

The group of roof nubs 906, 907, and 908 is composed of three design features that protrude from the lens cartridge's ceiling downward towards its floor. The shape, position, and protruding height of the three roof nubs correlate to specific areas of the IOL's rear haptic shoulder, IOL side optic edge, and IOL leading optic edge. The purpose of the group of the roof nubs 906, 907, and 908 is to secure the IOL's vertical position (Z direction) in a fixed position both during storage, transportation, or when injecting viscoelastic, where shifting of the IOL's vertical position may occur without these roof nub features. In some embodiments, lens cartridge 103 may comprise any combination of roof nubs 906, 907, and 908. For example, lens cartridge 103 may comprise one or more of roof nubs 906, 907, and 908 (e.g., roof nub 906 and 907 as illustrated at least with respect to FIGS. 9D and 9F).

The two curved walls 904 feature edges concentric to a circle of a predetermined size (e.g., about a 6 mm diameter in the illustrated example). In some embodiments, the curved walls 904 may be formed around a circle sized to correspond with (e.g., fit) the IOL. For example, the circle may be less than or equal to 6 mm, such as 5.5 mm, 5 mm, 4.5 mm, 4 mm, or less. In some embodiments, the circle may be greater than or equal to 6 mm, such as 6.5 mm, 7 mm, 7.5 mm, or more. Both the two curved walls 904 and the two straight walls 903 may have a height higher than the IOL's anterior surface when IOL is preloaded, as shown at least with respect to FIGS. 9G-9I. For example, the height of the curved walls 904 and straight walls 903 may be 0.2 mm, 0.3 mm, 0.4 mm, 0.5 mm, 0.6 mm, 0.7 mm, 0.8 mm, 0.9 mm, 1.0 mm, 1.1 mm, 1.2 mm, or more. The combination of the two curved walls 904 and the two straight walls 903 is intended to limit the lateral and longitudinal position of the IOL after it is preloaded, which provides a predictable position of the IOL after storage, transportation, or when injecting viscoelastic, where shifting of the IOL's position may occur. The straight walls 903 can limit the IOL from rotating in both clockwise and/or counterclockwise direction. The group of the roof nubs 906-908, two curved walls 904 and the two straight walls 903 can work together to limit the longitudinal, lateral, and vertical directions, as well as the rotations of the IOL.

In some embodiments, one or more plunge bumpers 905 can be located near the distal end of the lens cartridge 103 on either side of the plunger that facilitate the correction of the plunger's lateral (Y direction) position to ensure its starting position is collinear with the longitudinal axis (X direction) of the injector. The plunge bumpers 905 can be designed in a way that provides sufficient force to push against the plunger initially at an undeployed position, and elastic enough to bend away during the end of the plunger push travel towards the deployed position.

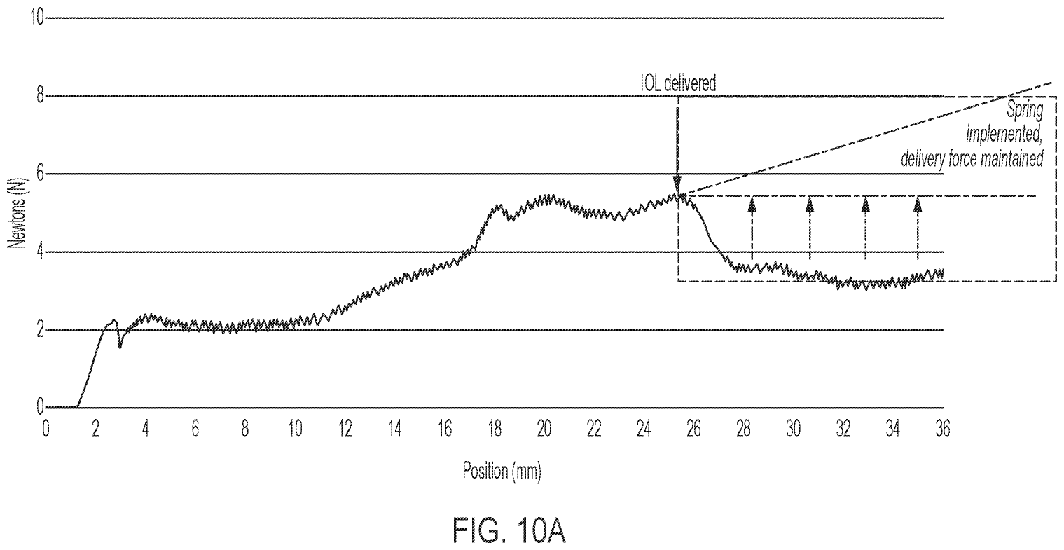

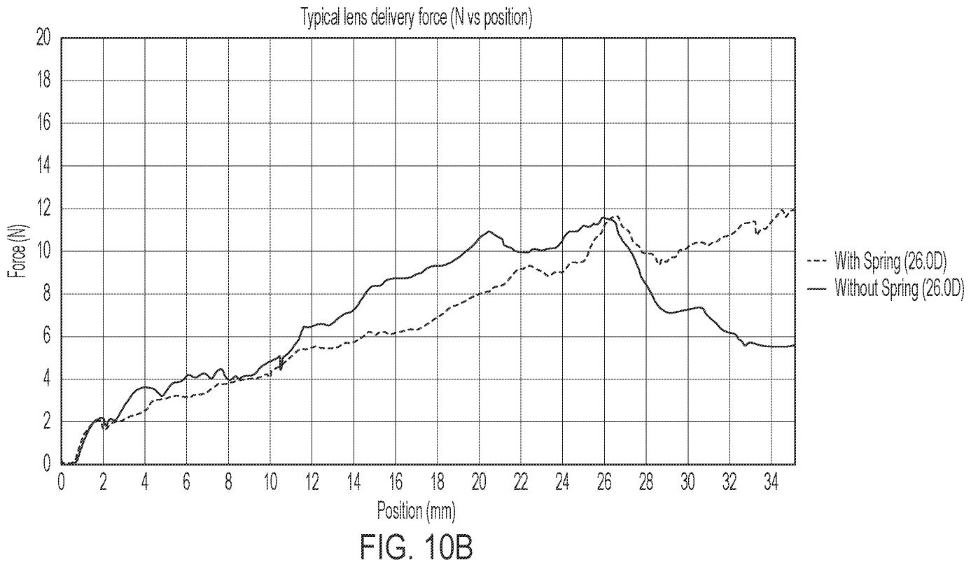

FIGS. 10A-10B illustrate typical lens delivery forces of the IOL injector versus the travel distance of the plunger, according to some embodiments of the present disclosure. In some embodiments, a spring can be incorporated to provide additional pushback force of the plunger onto the user's hand, which can limit the rapid decrease of force felt by the user after the moment an IOL is delivered and released from the distal end of the injector tip. FIG. 10B illustrates this difference in force against position for a lens injector with and without a spring, denoted accordingly. Typically, this sudden change of force can range from about 3 N-10 N and can often result in a sudden increase in plunger movement speed, which can cause the IOL to behave in an uncontrolled “shooting out” manner. Thus, by implementing a spring into the IOL injector, the force provided by the spring can be activated at the moment of release/injection, or right before the moment of IOL release, and last until the plunger is pushed to the very distal end of its travel. The result of the force graph shall be of a constant increase or maintenance of the peak force from the start until the end. Depending on the IOL's specifications, a specific spring may be matched with a particular IOL based on its design, brand, or diopter, to best achieve said force diagram result.

In some embodiments, the IOL injector is provided in a kit along with an IOL. The IOL may be preloaded in the lens cartridge of the injector, or in some embodiments provided separately from the IOL injector (i.e., external to the lens cartridge). Providing a preloaded IOL injector may reduce manual manipulation that is required by the physician in performing a surgical procedure, thereby reducing the risk of contamination and damage to the IOL which can occur during handling of the IOL. In some embodiments, providing the injector in a preloaded state allows that injector to be sterilized with the IOL simultaneously at the factory during packaging.

In some embodiments where the injector is preloaded, the lens injector can be provided with the IOL preloaded in the lens cartridge, whereby the lens is in an unfolded state, and the plunger can be in the undeployed state. Upon use, the physician simply needs to unpack the injector, gradually push the plunger from a proximal undeployed position towards the distal deployed position to fold and align the IOL in the lens cartridge, and deliver the IOL to the patient's eye. In some embodiments, the IOL injector may be configured for single use such that the injector is discarded after delivery of the IOL to the eye.

In some embodiments, the IOL injector may be provided without an IOL preloaded in the injector. For example, one or more IOLs may be provided separately from the IOL injector such that a user may manually load the IOL injector with an IOL. In some embodiments, the IOL injector (preloaded or not preloaded) may be sterilized and reused such that the IOL injector may be manually loaded for each subsequent use of the injector.

In some embodiments, a method of implanting an IOL into the eye of a patient is provided. The method includes providing an IOL injector of the disclosure having an IOL preloaded in the lens cartridge, depressing the plunger to fold and align the IOL in the lens cartridge lumen, and transitioning the plunger from the undeployed position to the deployed position to push the folded IOL along the longitudinal axis of the device and eject the IOL from the distal opening of the injector tip lumen into the eye of a patient.

In some embodiments, a method of implanting an IOL into the eye of a patient is provided. The method includes providing an IOL injector of the disclosure, inserting an IOL into the lens cartridge, depressing the plunger to fold and align the IOL in the lens cartridge lumen, and transitioning the plunger from the undeployed position to the deployed position to push the folded IOL along the longitudinal axis of the device and eject the IOL from the distal opening of the injector tip lumen into the eye of a patient.

In some embodiments, the lumen extending along the longitudinal axis of the injector need not have a constant diameter. For example, the lumen may comprise one or more diameters of about 1 mm, 1.5 mm, 2.0 mm, 2.5 mm, 3.0 mm, 3.5 mm, 4.0 mm, 4.5 mm, 5.0 mm, 5.5 mm, or 6.0 mm. In some embodiments, the lumen has a different diameter in different regions of the injector. For example, the lens cartridge lumen may have a different diameter than the injector tip lumen. In some embodiments, the diameter of the lens cartridge lumen is greater than the diameter of the injector tip lumen. In some embodiments, the injector tip lumen tapers from a larger diameter at the proximal end of the injector tip to a smaller diameter at the distal end of the injector tip where the distal opening is located.

In some embodiments, the cross-sectional area of the injector tip lumen decreases from a larger diameter at the proximal end of the injector tip to a smaller diameter at the distal end of the injector tip where the distal opening is located. Additionally, the cross-sectional shape of the injector tip lumen may be different at the distal end of the injector tip lumen as compared to the proximal end of the injector tip lumen.

In some embodiments, the distal opening of the disclosed IOL injector can have an elongated circular cross-sectional shape. The cross-sectional shape of the distal opening can be any shape necessary to maintain proper folding and orientation of the IOL for delivery to the eye. This may depend in part on the type and shape of the IOL being delivered. For example, the cross-sectional shape of the distal opening can be square, round, ellipse, rectangle, triangle, or curvilinear triangle.

The IOL injector may be constructed using a variety of different materials. One in the art will appreciate that different components of the injector may be constructed from different materials to impart different structural characteristics in different regions of the device. Further, various regions of the delivery lumen may include a polymer or lubricious coating.

Some embodiments of the present disclosure may provide IOL injectors with components constructed partially or entirely of polyurethane polymers; styrene related copolymers including but not limited to polyolefin, polyamide, PEBAX, acrylic butyldiene styrene (ABS), styrene butyldiene styrene (SBS), and/or high impact polystyrene (HIPS); polyester polymers; and polymeric blends or copolymers thereof. Such materials may have sufficient toughness to enable the creation of small diameter insertion devices and may also maintain or introduce other beneficial properties.

To ensure that the IOL is able to traverse the delivery lumen without causing damage to the lumen and/or the IOL, the delivery lumen should be able to withstand the application of the forces it will encounter during insertion. For example, the delivery lumen should be able to withstand forces up to at least 15 N. Accordingly, the delivery lumen may be formed from materials of sufficient toughness to withstand those forces without cracking or rupturing. In addition, to reduce the risk of damage to the IOL, and also reduce the insertion forces needed to perform the insertion, the delivery lumen may be formed of sufficiently lubricious material, be compounded with lubricating additives, be coated with a lubricating material, or otherwise minimize the forces tending to bind the IOL to the interior wall of the lumen. These considerations are especially true for the distal end of the injector tip since the tapering of the delivery lumen increases normal forces experienced by the delivery lumen as the IOL is moved distally.

In addition, to counter the frictional forces which may be experienced during the insertion process, in some embodiments a coating made of a hydrophilic polymeric material may be applied to the delivery lumen or lens cartridge to provide additional lubricity. For example, in some embodiments a coating may include one or more hydrophilic polymeric materials including but not limited to hydrophilic polyurethane, polyvinylpyrrolidone, polyacrylic acid, polyacrylamides, polyhydroxyethyl methacrylate, and/or hyaluronan, or the like. It is noted, however, that the lubricants used in the delivery lumen need not be applied uniformly along the lumen.

Additionally, some embodiments may utilize material formed using a co-molding process. Using such a process, two or more materials (e.g., polymers) may be extruded and/or injected to form a single piece and may allow for the use of materials having different physical properties. For instance, materials may be used having both sufficient toughness and other desirable properties. For instance, a polyurethane may be used having sufficient toughness, while another polyurethane may be used having desirable lubrication properties.

In some embodiments, the injector is constructed using polymer compositions having engrafted hydrophilic and lubricious groups as described in U.S. Pat. No. 10,494,458 to Lee, titled “Functionalized hydrophilic and lubricious polymeric matrix and methods of using same,” which is incorporated herein in its entirety by reference. Such compositions utilize polypropylene, polycarbonate, polyamide, cellulose acetate, and acrylic polymer or copolymer, which are suitable base polymers for engrafting.

In some embodiments, the IOLs can be evaluated for the optical properties, sagitta, and overall surface and bulk homogeneity before and after being surgically manipulated using the injector of the present disclosure. The injector of the present disclosure can also be evaluated for its cartridge and tip performance, such as overall cartridge and tip surface and bulk homogeneity. IOL optical properties and overall surface and bulk homogeneity inspection can be conducted in accordance with ISO 11979-2, Ophthalmic implants—Intraocular lenses—Part 2: Optical properties and test methods and ISO 11979-3, Ophthalmic implants—Intraocular lenses—Part 3: Mechanical properties and test methods.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The numerical ranges disclosed inherently support any range or value within the disclosed numerical ranges, including the endpoints, even though a precise range limitation is not stated verbatim in the specification because this disclosure can be practiced throughout the disclosed numerical ranges.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EMBODIMENTS

Embodiment 1. An intraocular lens (IOL) injector comprising:
an injector body elongated along a longitudinal direction, the injector body having a lumen disposed along the longitudinal direction;
a lens cartridge in operable connection with the injector body, the lens cartridge having a lumen configured to receive an IOL and coextensive with the injector body lumen, a cartridge cutout located along a lateral direction to house a leading haptic of the IOL, and a cartridge slope along the lateral direction to house a trailing haptic of the IOL;
an injector tip in operable connection with the lens cartridge, the injector tip having a lumen disposed along the longitudinal direction and terminating in a distal opening, wherein the injector tip lumen is coextensive with the injector body lumen; and
a plunger having an elongated shaft, the elongated shaft being slidably disposed within the injector body lumen from an undeployed position to a deployed position, wherein the plunger is configured to contact the IOL in the lens cartridge lumen, push the IOL along the longitudinal direction through the injector tip lumen to fold the IOL and inject the IOL out of the distal opening when the plunger is transitioned to the deployed position.

Embodiment 2. The IOL injector of embodiment 1, further comprising a label platform on the injector body.

Embodiment 3. The IOL injector of embodiment 1, further comprising a viewing window on top of the lens cartridge and the injector tip, adapted to provide an unobstructed view of the lens cartridge and the injector tip.

Embodiment 4. The IOL injector of embodiment 1, further comprising a finger hold flange on the injector body comprising contoured grip surfaces.

Embodiment 5. The IOL injector of embodiment 1, further comprising a proximal thumb press on the plunger, which is configured with contoured grip surfaces.

Embodiment 6. The IOL injector of embodiment 1, comprising an IOL preloaded into the lens cartridge.

Embodiment 7. The IOL injector of embodiment 1, wherein the lens cartridge comprises a plurality of roof nubs protruding from a ceiling to a floor of the lens cartridge, configured to maintain the position of the IOL in a vertical direction.

Embodiment 8. The IOL injector of embodiment 1, wherein the lens cartridge comprises two straight walls located at the lateral direction and two curved walls located at the lateral direction, configured to maintain the position of the IOL at the longitudinal and lateral directions.

Embodiment 9. The IOL injector of embodiment 1, wherein the plunger comprises a distal tip comprising a curved surface, a middle cutout, and a lateral cutout.

Embodiment 10. The IOL injector of embodiment 1, wherein a surface of the injector tip lumen or the lens cartridge lumen comprises a polymer or lubricious coating.

Embodiment 11. The IOL injector of embodiment 10, wherein the lubricious coating is a hydrophilic and lubricious polymer or copolymer matrix.

Embodiment 12. The IOL injector of embodiment 1, wherein the distal opening of the injector tip lumen is a geometric shape selected from the group consisting of square, round, ellipse, rectangle, triangle, and curvilinear triangle.

Embodiment 13. The IOL injector of embodiment 1, wherein the injector tip lumen has a proximal region having an opening, wherein the cross-sectional area of the opening is greater than the cross-section area of the distal opening.

Embodiment 14. A method of implanting an intraocular lens (IOL), comprising:
(a) providing an IOL injector pre-loaded with an IOL, the injector comprising:
   (i) an injector body elongated along a longitudinal direction, the injector body having a lumen disposed along the longitudinal direction;
   (ii) a lens cartridge pre-loaded with the IOL in operable connection with the injector body, the lens cartridge having a lumen configured to receive an IOL and coextensive with the injector body lumen, a cartridge cutout located along a lateral direction to house a leading haptic of an IOL, and a cartridge slope along the lateral direction to house a trailing haptic of the IOL;
   (iii) an injector tip in operable connection with the lens cartridge, the injector tip having a lumen disposed along the longitudinal direction and terminating in a distal opening, wherein the injector tip lumen is coextensive with the injector body lumen; and
   (iv) a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position;
(b) inserting an IOL into the lens cartridge lumen; and
(c) transitioning the plunger from the undeployed position to the deployed position thereby ejecting the IOL from the distal opening of the injector tip lumen into the eye of a patient, the IOL being in a folded state while exiting the distal opening.

Embodiment 15. The method of embodiment 14, further comprising adding a fluid into the lens cartridge prior to inserting the IOL.

Embodiment 16. The method of embodiment 14, wherein the fluid is selected from water, viscoelastic, and balanced salt solution (BSS).

Embodiment 17. A kit comprising the IOL injector of embodiment 1 and an IOL.

Embodiment 18. The kit of embodiment 17, wherein the IOL comprises an optic and at least one haptic extending in a curved shape from the optic.

Embodiment 19. The kit of embodiment 17, wherein the injector is preloaded with the IOL.

Embodiment 20. The kit of embodiment 17, further comprising a fluid selected from water, viscoelastic, and balanced salt solution (BSS).

Embodiment 21. An intraocular lens (IOL) injector comprising:
an injector body elongated along a longitudinal direction, the injector body having an injector body lumen disposed along the longitudinal direction;
a lens cartridge in operable connection with the injector body, the lens cartridge having a lens cartridge lumen configured to receive an IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
an injector tip in operable connection with the lens cartridge, the injector tip having an injector tip lumen disposed along the longitudinal direction and terminating in a distal opening, wherein the injector tip lumen is coaxial with the injector body lumen; and
a plunger having an elongated shaft, the elongated shaft being slidably disposed within the injector body lumen from an undeployed position to a deployed position, wherein the plunger is configured to contact the IOL in the lens cartridge lumen, push the IOL along the longitudinal direction through the injector tip lumen to fold the IOL and inject the IOL out of the distal opening when the plunger is transitioned to the deployed position.

Embodiment 22. The IOL injector of embodiment 21, comprising a label platform on the injector body.

Embodiment 23. The IOL injector of embodiment 21, comprising a viewing window adjacent to the lens cartridge and the injector tip, the viewing window adapted to provide a view of the lens cartridge and the injector tip.

Embodiment 24. The IOL injector of embodiment 21, comprising a finger hold flange on the injector body, the finger hold flange comprising contoured grip surfaces.

Embodiment 25. The IOL injector of embodiment 21, comprising a proximal thumb press on the plunger, the proximal thumb press comprising contoured grip surfaces.

Embodiment 26. The IOL injector of embodiment 21, comprising an IOL preloaded into the lens cartridge.

Embodiment 27. The IOL injector of embodiment 21, wherein the lens cartridge comprises a plurality of roof nubs protruding from a ceiling to a floor of the lens cartridge, the roof nubs configured to maintain the position of the IOL in a vertical direction.

Embodiment 28. The IOL injector of embodiment 21, wherein the lens cartridge comprises two straight walls located at the lateral direction and two curved walls located at the lateral direction, the two straight walls and the two curved walls configured to maintain the position of the IOL at the longitudinal direction and the lateral direction.

Embodiment 29. The IOL injector of embodiment 21, wherein the plunger comprises a distal tip comprising a curved surface, a middle cutout, and a lateral cutout.

Embodiment 30. The IOL injector of embodiment 21, wherein a surface of the injector tip lumen or the lens cartridge lumen comprises a polymer or lubricious coating.

Embodiment 31. The IOL injector of embodiment 30, wherein the lubricious coating is a hydrophilic and lubricious polymer or copolymer matrix.

Embodiment 32. The IOL injector of embodiment 21, wherein the distal opening of the injector tip lumen is a geometric shape selected from the group consisting of: square, round, ellipse, rectangle, triangle, and curvilinear triangle.

Embodiment 33. The IOL injector of embodiment 21, wherein the injector tip lumen has a proximal region having an opening, wherein a cross-sectional area of the proximal opening is greater than a cross-section area of the distal opening.

Embodiment 34. A method of implanting an intraocular lens (IOL), comprising:
(a) providing an IOL injector preloaded with an IOL, the injector comprising:
   (i) an injector body elongated along a longitudinal direction, the injector body having an injector body lumen disposed along the longitudinal direction;
   (ii) a lens cartridge preloaded with the IOL and in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
   (iii) an injector tip in operable connection with the lens cartridge, the injector tip having an injector tip lumen disposed along the longitudinal direction and terminating in a distal opening, wherein the injector tip lumen is coaxial with the injector body lumen; and
   (iv) a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position; and
(b) transitioning the plunger from the undeployed position to the deployed position, thereby ejecting the IOL from the distal opening of the injector tip lumen into an eye of a patient, the IOL being in a folded state while exiting the distal opening.

Embodiment 35. The method of embodiment 34, wherein the lens cartridge comprises a fluid selected from the group consisting of: water, viscoelastic, and balanced salt solution (BSS).

Embodiment 36. A method of implanting an intraocular lens (IOL), comprising:
(a) providing an IOL injector, the injector comprising:
   (i) an injector body elongated along a longitudinal direction, the injector body having an injector body lumen disposed along the longitudinal direction;
   (ii) a lens cartridge in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
   (iii) an injector tip in operable connection with the lens cartridge, the injector tip having an injector tip lumen disposed along the longitudinal direction and terminating in a distal opening, wherein the injector tip lumen is coaxial with the injector body lumen; and
   (iv) a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position;
(b) inserting the IOL into the lens cartridge lumen; and
(c) transitioning the plunger from the undeployed position to the deployed position, thereby ejecting the IOL from the distal opening of the injector tip lumen into an eye of a patient, the IOL being in a folded state while exiting the distal opening.

Embodiment 37. The method of embodiment 36, comprising adding a fluid into the lens cartridge prior to inserting the IOL.

Embodiment 38. The method of embodiment 37, wherein the fluid is selected from the group consisting of: water, viscoelastic, and balanced salt solution (BSS).

Embodiment 39. A kit comprising the IOL injector of embodiment 1 and an IOL.

Embodiment 40. The kit of embodiment 39, wherein the IOL comprises an optic and at least one haptic extending in a curved shape from the optic.

Embodiment 41. The kit of embodiment 39, wherein the injector is preloaded with the IOL.

Embodiment 42. The kit of embodiment 39, wherein the lens cartridge comprises a fluid selected from the group consisting of: water, viscoelastic, and balanced salt solution (BSS).

1. An intraocular lens (IOL) injector comprising:
an injector body elongated along a longitudinal direction, the injector body having an injector body lumen disposed along the longitudinal direction;
a lens cartridge in operable connection with the injector body, the lens cartridge having a lens cartridge lumen configured to receive an IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
an injector tip in operable connection with the lens cartridge, the injector tip having an injector tip lumen disposed along the longitudinal direction and terminating in a distal opening, wherein the injector tip lumen is coaxial with the injector body lumen; and
a plunger having an elongated shaft, the elongated shaft being slidably disposed within the injector body lumen from an undeployed position to a deployed position, wherein the plunger is configured to contact the IOL in the lens cartridge lumen, push the IOL along the longitudinal direction through the injector tip lumen to fold the IOL and inject the IOL out of the distal opening when the plunger is transitioned to the deployed position.

2. The IOL injector of claim 1, comprising a label platform on the injector body.

3. The IOL injector of claim 1, comprising a viewing window adjacent to the lens cartridge and the injector tip, the viewing window adapted to provide a view of the lens cartridge and the injector tip.

4. The IOL injector of claim 1, comprising a finger hold flange on the injector body, the finger hold flange comprising contoured grip surfaces.

5. The IOL injector of claim 1, comprising a proximal thumb press on the plunger, the proximal thumb press comprising contoured grip surfaces.

6. The IOL injector of claim 1, comprising an IOL preloaded into the lens cartridge.

7. The IOL injector of claim 1, wherein the lens cartridge comprises a plurality of roof nubs protruding from a ceiling to a floor of the lens cartridge, the roof nubs configured to maintain the position of the IOL in a vertical direction.

8. The IOL injector of claim 1, wherein the lens cartridge comprises two straight walls located at the lateral direction and two curved walls located at the lateral direction, the two straight walls and the two curved walls configured to maintain the position of the IOL at the longitudinal direction and the lateral direction.

9. The IOL injector of claim 1, wherein the plunger comprises a distal tip comprising a curved surface, a middle cutout, and a lateral cutout.

10. The IOL injector of claim 1, wherein a surface of the injector tip lumen or the lens cartridge lumen comprises a polymer or lubricious coating.

11. The IOL injector of claim 10, wherein the lubricious coating is a hydrophilic and lubricious polymer or copolymer matrix.

12. The IOL injector of claim 1, wherein the distal opening of the injector tip lumen is a geometric shape selected from the group consisting of: square, round, ellipse, rectangle, triangle, and curvilinear triangle.

13. The IOL injector of claim 1, wherein the injector tip lumen has a proximal region having an opening, wherein a cross-sectional area of the proximal opening is greater than a cross-section area of the distal opening.

14. A method of implanting an intraocular lens (IOL), comprising:
(a) providing an IOL injector preloaded with an IOL, the injector comprising:
(i) an injector body elongated along a longitudinal direction, the injector body having an injector body lumen disposed along the longitudinal direction;
(ii) a lens cartridge preloaded with the IOL and in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
(iii) an injector tip in operable connection with the lens cartridge, the injector tip having an injector tip lumen disposed along the longitudinal direction and terminating in a distal opening, wherein the injector tip lumen is coaxial with the injector body lumen; and
(iv) a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position; and
(b) transitioning the plunger from the undeployed position to the deployed position, thereby ejecting the IOL from the distal opening of the injector tip lumen into an eye of a patient, the IOL being in a folded state while exiting the distal opening.

15. The method of claim 14, wherein the lens cartridge comprises a fluid selected from the group consisting of: water, viscoelastic, and balanced salt solution (BSS).

16. A method of implanting an intraocular lens (IOL), comprising:
(a) providing an IOL injector, the injector comprising:
(i) an injector body elongated along a longitudinal direction, the injector body having an injector body lumen disposed along the longitudinal direction;
(ii) a lens cartridge in operable connection with the injector body, the lens cartridge having a lens cartridge lumen configured to receive the IOL, wherein the lens cartridge lumen is coaxial with the injector body lumen, a cartridge cutout located along a lateral direction transverse to the longitudinal direction and configured to house a leading haptic of the IOL, and a cartridge slope located along the lateral direction and configured to house a trailing haptic of the IOL;
(iii) an injector tip in operable connection with the lens cartridge, the injector tip having an injector tip lumen disposed along the longitudinal direction and terminating in a distal opening, wherein the injector tip lumen is coaxial with the injector body lumen; and
(iv) a plunger having an elongated shaft, the elongated shaft being slidably disposed within the injector body lumen from an undeployed position to a deployed position, wherein the plunger is configured to contact the IOL in the lens cartridge lumen and push the IOL along the longitudinal direction through the injector tip lumen and out of the distal opening when the plunger is transitioned to the deployed position;
(b) inserting the IOL into the lens cartridge lumen; and
(c) transitioning the plunger from the undeployed position to the deployed position, thereby ejecting the IOL from the distal opening of the injector tip lumen into an eye of a patient, the IOL being in a folded state while exiting the distal opening.

17. The method of claim 16, comprising adding a fluid into the lens cartridge prior to inserting the IOL.

18. The method of claim 16, wherein the fluid is selected from the group consisting of: water, viscoelastic, and balanced salt solution (BSS).

19. A kit comprising the IOL injector of claim 1 and an IOL.

20. The kit of claim 19, wherein the IOL comprises an optic and at least one haptic extending in a curved shape from the optic.

21. The kit of claim 19, wherein the injector is preloaded with the IOL.

22. The kit of claim 19, wherein the lens cartridge comprises a fluid selected from the group consisting of: water, viscoelastic, and balanced salt solution (BSS).
